(19) United States
(12) Patent Application Publication
(10) Pub. No.: US 2015/0080613 A1
(43) Pub. Date: Mar. 19, 2015

(54) METHOD FOR PREPARING GLYCEROL ETHER AND GLYCOL ETHER

(71) Applicants: RHODIA OPERATIONS, Paris (FR); CENTRE NATIONAL DE LA RECHERCHE SCIENTIQUE (C.N.R.S), Paris (FR); Université Claude Bernard Lyon 1, Villeurbanne (FR); FONDS DE DEVELOPPEMENT DES FILIERES DES OLEAGINEUX ET PROTEAGINEUX FIDOP, Paris (FR)

(72) Inventors: Gérard Mignani, Lyon (FR); Marc Lemaire, Villeurbanne (FR); Eric Da Silva, Lyon (FR); Wissam Dayoub, Villeurbanne (FR); Yann Raoul, Crezancy (FR)

(21) Appl. No.: 14/386,877

(22) PCT Filed: Mar. 25, 2013
(86) PCT No.: PCT/EP2013/056280

§ 371 Date: Sep. 22, 2014

(30) Foreign Application Priority Data
Mar. 23, 2012 (FR) 1252624

(51) Int. Cl. C07C 41/01

(52) U.S. Cl. CPC C07C 41/01
USPC 568/680; 568/678

(57) ABSTRACT

The present invention concerns a method for preparing glycerol ether or glycol ether comprising the reaction of a compound of formula (II) with a compound of formula (III) in the presence of a heterogeneous acid catalyst of formulas (II) and (III).

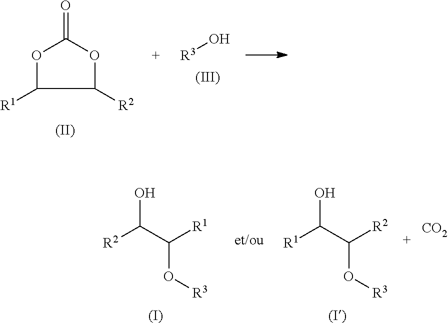

This invention relates to a method for preparing glycerol ether and glycol ether.

Glycerol and its derivatives are significant industrial by-products, in particular in the biodiesel fuel industry. It is therefore particularly beneficial to find new ways to upgrade these products.

Glycerol ethers and glycol ethers can be used in numerous fields, such as cosmetics, detergents, washing formulations and in the pharmaceutical field. These ethers can constitute a new line of particularly beneficial surfactants since they are obtained from biosourced materials. However, there are few synthesis methods enabling these ethers to be obtained in a simple and inexpensive manner.

From JP 200-119205 in particular, a method for producing glycerol ether from glycerol carbonate by reaction in the presence of a base (in particular KOH) is known. However, the use of this method does not enable a good glycerol ether yield to be obtained; indeed, the transcarbonation compound is primarily formed.

The objective of this invention is to provide a method for selective preparation of glycerol ether or derivatives of glycerol and glycol ether.

Another objective of this invention is to provide such a method that makes it possible to obtain, with good yields, the desired ether.

Another objective of this invention is to provide a method for preparing surfactants from biosourced compounds.

Another objective is to provide a continuous method for preparing glycerol ether or derivatives of glycerol and glycol ether.

This invention relates to a method for preparing glycerol ether or glycol ether of formula (I) and/or (I′), comprising the reaction of a compound of formula (II) with a compound of formula (III) in the presence of a heterogeneous acid catalyst

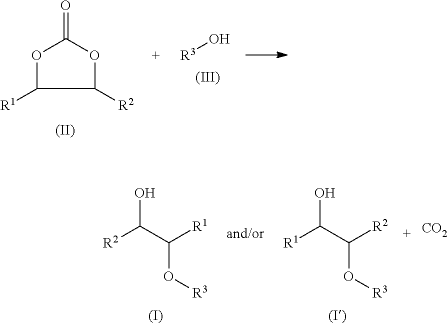

wherein

R¹ is a hydrogen atom or an alkyl radical, linear or branched, comprising 1 to 15 carbon atoms, preferably 1 to 10 carbon atoms;

R² is a hydrogen atom; an alkyl radical, linear or branched, comprising 1 to 15 carbon atoms, preferably 1 to 10 carbon atoms; or a group of formula —(CH₂)ₙOH, wherein n is an integer between 0 and 5, and n is preferably equal to 0 or 1;

R³ is an alkyl radical, linear or branched, capable of comprising one or more unsaturations, comprising 1 to 40 carbon atoms, and optionally capable of comprising 1 or more hydroxy substituents (OH).

In the context of the invention, it is possible to selectively obtain one of compounds (I) or (I′) or a mixture of these two compounds according to the definition of the R¹ and R² groups, the steric hindrance orienting the reaction toward the addition of the OR³ group on the less hindered side. Thus, without being bound to any one theory, preferably:
when R¹ is H or an alkyl radical as defined above and R² is a group of formula —(CH₂)ₙOH, as defined above, the reaction leads to the formation of compound I;
when R¹ is H and R² is an alkyl radical as defined above, the reaction leads to the formation of a mixture of compounds (I) and (I′), compound (I) being capable of being present in the majority;
when R² is H and R¹ is an alkyl radical as defined above, the reaction leads to the formation of a mixture of compounds (I) and (I′), compound (I′) being capable of being present in the majority;
when R¹ is an alkyl radical as defined above and R² is an alkyl radical as defined above, the reaction leads to the formation of a mixture of compounds (I) and (I′), and the compound resulting from the addition to the carbon bearing the group comprising the fewest carbon atoms can be obtained in a majority.

Preferably, in the method of the invention R¹ is a hydrogen atom, R² is —CH₂OH, compound (II) thus preferred is glycerol carbonate, and the compound formed is a compound of formula (I):

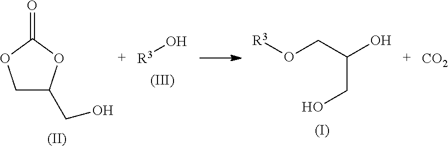

Preferably, in the method of the invention, R³ is an alkyl radical, linear or branched, optionally comprising one or more unsaturations, comprising 1 to 40 carbon atoms. In a preferred embodiment, R³ is an alkyl radical, linear or branched, optionally comprising one or more unsaturations, comprising 12 to 40 carbon atoms, preferably 24 to 30 carbon atoms. In another preferred embodiment R^{3 is} an alkyl radical, linear or branched, optionally comprising one or more unsaturations, comprising 1 to 15 carbon atoms.

In one embodiment, the method relates to the preparation of glycerol ether.

In another embodiment, the method relates to the preparation of glycol ether.

The features described below apply to each of these two embodiments.

Advantageously, the heterogeneous acid catalyst of the invention has an acid site concentration greater than or equal to 0.01 mequi/g (or mEq/g or me/g) (milliequivalent of H⁺ ions per gram) of catalyst, preferably 0.01 to 10 mequi/g (or mEq/g or me/g), more preferably 0.01 to 6 mequi/g (or mEq/g or me/g), preferably 0.01 to 5 mequi/g (or mEq/g or me/g). This makes it possible in particular to obtain a good conversion of the compound of formula (II) and a good ether yield (compound of formula (I) or (I′)). Advantageously, the greater the acid site concentration is, the higher the glycerol ether yield will be.

In the context of this invention, the term “acid site concentration” refers to the surface acidity due to H⁺ protons at the surface of the catalyst. This acid site concentration is determined by any method known to a person skilled in the art, and in particular usually by determining the milliequivalent number of H⁻ protons brought to 1 g of catalyst (mequi/g (mEq/g or me/g) of catalyst). The acid site concentration in mEq/g corresponds to the ion exchange capacity of the catalyst expressed in mEq of H⁻ per gram of catalyst.

Advantageously, the heterogeneous catalyst according to the invention has a specific surface measured by the BET method of 5 to 500 m²/g, preferably 10 to 100 m²/g. The specific surface is determined by the BET method, for example by the nitrogen adsorption and desorption method.

This makes it possible in particular to obtain a good conversion of the compound of formula (II) and a good ether yield. Advantageously, the greater the specific surface is, the higher the glycerol ether or glycol ether yield will be.

Preferably, the heterogeneous catalyst according to the invention is characterized by a Hammett constant (Ho) of −3 to −12, preferably −5 to −12. This makes it possible, in particular, to obtain a good conversion of the compound of formula (II) and a good ether yield. Advantageously, the lower the Ho is, the higher the glycerol ether or glycol ether yield will be.

The Hammett constant can be determined by any method known to a person skilled in the art and is in particular determined by a standardized colorimetric method called the Tanabe method (TANABE et al. The Journal of Physical Chemistry, (1976) 15, 1723).

The acid catalyst AH is reacted with a colour indicator B, the reaction leads to the formation of A⁻ and BH⁺. The Ho value is then determined by the formula (A):

$$Ho = pKa ( {BH}^{+} / B ) + log ( \frac{[ B ]}{[ {BH}^{+} ]} ) \qquad ( A )$$

In this formula, pKa(BH⁺/B) is the pKa of the acid/base pair (BH⁺/B); [B] is the concentration of B and [BH⁻] is the concentration of BH⁻.

Preferably, the catalyst is chosen from the group consisting of the acid forms of ion exchange resins; supports impregnated with sulphuric acid, hydrochloric acid, niobic acid, hydrofluoric acid, antimony pentafluoride, heteropoly acids, triflic acid, or sulfonic or phosphoric acid; sulphated zirconia; zeolites, in particular aluminosilicate zeolite, for example zeolite Y characterized by a faujasite structure; and mixed oxides, in particular TiO₂/Al₂O₃, ReO₇/Al₂O₃, TiO₂/ZrO₂, SiO₂/Al₂O3.

The supports are in particular chosen from metal oxides, in particular Al₂O₃, ZrO₂, TiO₂; SiO₂; or carbons.

In an especially preferred manner, the catalyst is chosen from the acid forms of ion exchange resins; supports impregnated with sulphuric acid or sulfonic acid; and sulphated zirconia.

Even more preferably, the heterogeneous catalyst is chosen from the acid forms of ion exchange resins.

All of the preferred or advantageous features of the acid catalyst according to the invention can be combined with one another.

The ion exchange acid resins can in particular be chosen from the ion exchange acid resins bearing sulfonic groups. They may in particular be chosen from the resins consisting of a polystyrene skeleton bearing sulfonic groups or from the perfluorinated resins bearing sulfonic groups.

Preferably, the resins consisting of a polystyrene skeleton are styrene-divinylbenzene copolymers comprising sulfonic groups. Such a resin is obtained by polymerization of the styrene and the divinylbenzene under the influence of an activation catalyst, usually in suspension. Beads or granules are obtained, which are then treated with concentrated sulphuric or chlorosulphuric acid. The proportion of the sulfonic groups with respect to the polymeric mass can be variable, and will be taken into account when determining the amount of polymer to use. Such resins are in particular commercially available under the name Amberlyst® (sold by the Dow company). Preferably, these resins are chosen from Amberlyst® 35, Amberlyst® 36, Amberlyst® 70 or Amberlyst® 21.

Preferably, the perfluorinated resins bearing sulfonic groups are tetrafluoroethylene and perfluoro[2-(fluorosulfonyl-ethoxy)-propyl]vinyl ether copolymers, in particular those sold under the name Naflon®. These resins have the following formula:

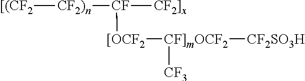

wherein m is an integer with a value of 1, 2 or 3, n is an integer from 5 to 13 and x is generally around 1000.

Particularly preferably, the resins are resins consisting of a polystyrene skeleton bearing sulfonic groups.

Preferably, the catalyst is used in proportions of 2% to 40%, preferably 5% to 20% by weight with respect to the weight of the compound of formula (II).

Preferably, the molar ratio of formula (II) compound/formula (III) compound is 1/1 to 1/5, and preferably 1/2 to 1/4.

It is preferable, in the context of the method of the invention, to control the amount of water introduced by the different reagents. It is thus preferable to dry the reagents before use.

The maximum temperature used in the method of the invention is primarily dependent upon the acid used. In fact, certain resins are sensitive to temperature. A person skilled in the art can therefore adapt the temperature of the method to the acid used. Preferably, the method of the invention can be implemented at a temperature of 100° C. to 200° C., preferably 100° C. to 170° C., for example 100° C. to 150° C.

The duration of the method of this invention can be 30 minutes to 24 hours, and preferably 30 minutes to 12 hours.

The method of the invention can be performed in batch or continuous mode, and is preferably performed in continuous mode.

The method of the invention advantageously makes it possible to obtain glycerol ethers with a purity greater than or equal to 90%, preferably greater than or equal to 99%.

Particularly advantageously, when the compound of formula (III) is a fatty alcohol, i.e. when R³ is an alkyl, linear or branched, comprising 12 to 40 carbon atoms, preferably 24 to 30 carbon atoms, the ethers thus obtained may in particular be used as surfactants. These ethers will in particular advantageously be capable of being used as surfactants in detergent compositions, cosmetic compositions, washing formulations and in the pharmaceutical field.

According to the invention, the method may comprise a preliminary step of preparing the compound of formula (II). This preliminary step is performed by a reaction between a compound of formula (IV) and carbon dioxide, in the presence of a lanthanide-based catalyst:

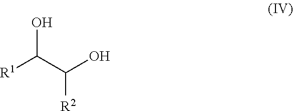

wherein R¹ and R² are as defined for formula (I).

In one embodiment, the lanthanide-based catalyst is chosen from the lanthanide family, and more specifically from the rare earth group, supported or unsupported.

The term “rare earth” (designated throughout the description by the generic term Ln) refers to chemical elements chosen from the group consisting of cerium (Ce), lanthanum (La), praseodymium (Pr), neodymium (Nd), yttrium (Y), gadolinium (Gd), samarium (Sm) and holmium (Ho), alone or in a mixture, preferably cerium, lanthanum, praseodymium and neodymium, alone or in a mixture.

In one embodiment, the catalyst is chosen from the group consisting of lanthanide oxides of formula Ln₂O₃ (for lanthanum, neodymium, yttrium, gadolinium, samarium and holmium) or CeO₂ or Pr₆O₁₁, lanthanide carbonates of formula Ln₂(CO₃)₃, lanthanide hydroxycarbonates of formula Ln(OH)(CO₃), lanthanide oxycarbonates of formula Ln₂O(CO₃)₂ and lanthanide hydroxides of formula Ln(OH)₃, alone or in a mixture.

In a preferred embodiment, the catalyst is chosen from the group consisting of lanthanide oxides, lanthanide carbonates and lanthanide hydroxycarbonates, alone or in a mixture; preferably, the catalyst is chosen from the group consisting of lanthanide oxides, or lanthanide carbonates, alone or in a mixture.

In one embodiment, the catalyst is a rare earth oxide.

In one embodiment, the catalyst of the preliminary step is chosen from the group consisting of CeO₂ and Pr₆O₁₁.

In one embodiment, the catalyst of the preliminary step is in oxide form and has a specific surface of at least 5 m²/g, preferably at least 10 m²/g, and more preferably at least 30 m²/g.

In one embodiment of the invention, the catalyst of the preliminary step, as defined above, is doped by Lewis acid-type metals, for example transition metals, alkaline earth metals and metalloids.

In one embodiment, these metals are chosen from the group consisting of iron (Fe(II) and Fe(III)), copper (Cu(I) and Cu(III)), aluminium (Al(III)), titanium (Ti(IV)), boron (B(III)), zinc (Zn(II)) and magnesium (Mg(II)).

Preferably, these metals are chosen from the group consisting of iron (Fe(II) and Fe(III)), copper (Cu(I) and Cu(III)), titanium (Ti(IV)) and zinc (Zn(II)).

In one embodiment, the catalyst is a rare earth oxide modified with transition metals.

In this embodiment, the relative percentage of metals with respect to the lanthanide material is between 1 and 10% by weight, and preferably between 1 and 5% by weight.

In one embodiment of the invention, in order to minimize costs, the catalyst may be a mixed system based on rare earths and other minerals such as ZnO, MgO, Al₂O₃ or SiO₂.

This particular embodiment makes it possible to provide additional properties in terms of both the acid-basic properties and the mechanical properties of the catalysts.

Advantageously, the molar ratio between the compound of formula (IV) and CO₂ is between 1 and 150 molar equivalents, preferably between 1 and 100 equivalents.

According to the invention, the preliminary step of preparing the compound of formula (II) is implemented at autogenous pressure or at atmospheric pressure.

According to the invention, the preliminary step of preparing the compound of formula (II) is implemented at a temperature of between 25 and 250° C., preferably between 25 and 200° C., for example between 50 and 150° C.

Advantageously, the amount of catalyst is between 0.01 and 50% by weight with respect to the weight of the compound of formula (IV), preferably between 1 and 25% by weight, and preferably between 3 and 15% by weight.

This invention will now be described with the assistance of non-limiting examples.

### EXAMPLE 1

### Synthesis of Glycerol 1-O-Octylether by Acid Catalysis from Glycerol Carbonate

In a 50-mL three-necked flask, provided with a coolant and a nitrogen inlet, 5.20 g (40 mmol) of commercial n-octanol and 118 mg of Amberlyst A 35 solid acid are introduced at room temperature. The reaction medium is then brought to 140° C. and 118 mg (10 mmol) of glycerol carbonate are added over a period of one hour. The heating at 140° C. is then extended for 1 h after the end of the addition. Then, the reaction medium is brought to room temperature. Then, 20 mL of CH₂Cl₂ as well as 5 mL of H₂O are added. The organic phase is then decanted. The aqueous phase is extracted by 2×25 mL of CH₂Cl₂. The organic phases are combined and the CH₂Cl₂ is evaporated under reduced pressure. The crude reaction product is then purified by flash silica column chromatography (Eluent (AcOEt/cyclohexane: 1/4 to 1/1)) to give glycerol 1-O-octylether with an isolated yield of 45%.

### EXAMPLE 2

### Synthesis of Glycerol 1-O-Decylether by Acid Catalysis from Glycerol Carbonate

In a 50-mL three-necked flask, provided with a coolant and a nitrogen inlet, 6.33 g (40 mmol) of commercial n-decanol and 118 mg of Amberlyst A 35 solid acid are introduced at room temperature. The reaction medium is then brought to 140° C. and 118 mg (10 mmol) of glycerol carbonate are added over a period of one hour. The heating at 140° C. is then extended for 1 h after the end of the addition. Then, the reaction medium is brought to room temperature. Then, 20 mL of CH₂Cl₂ as well as 5 mL of H₂O are added. The organic phase is then decanted.

The aqueous phase is extracted by 2×25 mL of CH₂Cl₂. The organic phases are combined and the CH₂Cl₂ is evaporated under reduced pressure. The crude reaction product is then purified by flash silica column chromatography (Eluent (AcOEt/cyclohexane: 1/4 to 1/1)) to give glycerol 1-O-decylether with an isolated yield of 46%.

### EXAMPLE 3

### Synthesis of Glycerol 1-O-Dodecylether by Acid Catalysis from Glycerol Carbonate

In a 50-mL three-necked flask, provided with a coolant and a nitrogen inlet, 7.44 g (40 mmol) of commercial n-dodecanol and 118 mg of Amberlyst A 35 solid acid are introduced at room temperature. The reaction medium is then brought to 140° C. and 118 mg (10 mmol) of glycerol carbonate are added over a period of one hour. The heating at 140° C. is then extended for 1 h after the end of the addition. Then, the reaction medium is brought to room temperature. Then, 20 mL of CH₂Cl₂ as well as 5 mL of H₂O are added. The organic phase is then decanted. The aqueous phase is extracted by 2×25 mL of CH₂Cl₂. The organic phases are combined and the CH₂Cl₂ is evaporated under reduced pressure. The crude reaction product is then purified by flash silica column chromatography (Eluent (AcOEt/cyclohexane: 1/4 to 1/1)) to give glycerol 1-O-dodecylether with an isolated yield of 40%.

### EXAMPLE 4

### Synthesis of Glycerol 1-O-Octylether by Acid Catalysis from Glycerol Carbonate

In a 50-mL three-necked flask, provided with a coolant and a nitrogen inlet, 5.20 g (40 mmol) of commercial n-octanol and 118 mg of Amberlyst A 36 solid acid are introduced at room temperature. The reaction medium is then brought to 140° C. and 118 mg (10 mmol) of glycerol carbonate are added over a period of one hour. The heating at 140° C. is then extended for 1 h after the end of the addition. Then, the reaction medium is brought to room temperature. Then, 20 mL of CH₂Cl₂ as well as 5 mL of H₂O are added. The organic phase is then decanted. The aqueous phase is extracted by 2×25 mL of CH₂Cl₂. The organic phases are combined and the CH₂Cl₂ is evaporated under reduced pressure. The crude reaction product is analysed without purification by gas phase chromatography. Glycerol octyl ether is detected with a GC yield of 15%.

### EXAMPLE 5

### Synthesis of Glycol 1-O-Octyléther by Acid Catalysis from Propylene Carbonate

In a 50-mL three-necked flask, provided with a coolant and a nitrogen inlet, 5.20 g (40 mmol) of commercial n-octanol and 118 mg of Amberlyst A 35 solid acid are introduced at room temperature. The reaction medium is then brought to 140° C. and 1.02 g (10 mmol) of propylene carbonate are added over a period of one hour. The heating at 140° C. is then extended for 1 h after the end of the addition. Then, the reaction medium is brought to room temperature. Then, 20 mL of CH₂Cl₂ as well as 5 mL of H₂O are added. The organic phase is then decanted. The aqueous phase is extracted by 2×25 mL of CH₂Cl₂. The organic phases are combined and the CH₂Cl₂ is evaporated under reduced pressure. The crude reaction product is analysed without purification by gas phase chromatography. Octyl propylene glycol ether is detected with a GC yield of 85% for a conversion of 90%.

### EXAMPLE 6

### Synthesis of Glycerol 1-O-Pentyl Ether by Acid Catalysis from Glycerol Carbonate

In a 25-mL three-necked flask, provided with a coolant and a nitrogen inlet, 3.53 g (40 mmol) of commercial pentan-1-ol and 118 mg of Amberlyst A 35 solid acid are introduced at room temperature. The reaction medium is then brought to 140° C. and 1.18 g (10 mmol) of glycerol carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 1/4 to 1/1)) to give glycerol 1-O-pentyl ether with an isolated yield of 49%.

### EXAMPLE 7

### Synthesis of Glycerol 1-O-Heptyl Ether by Acid Catalysis from Glycerol Carbonate

In a 25-mL three-necked flask, provided with a coolant and a nitrogen inlet, 4.65 g (40 mmol) of commercial heptan-1-ol and 118 mg of Amberlyst A 35 solid acid are introduced at room temperature. The reaction medium is then brought to 140° C. and 1.18 g (10 mmol) of glycerol carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 1/4 to 1/1)) to give glycerol 1-O-heptyl ether with an isolated yield of 42%.

### EXAMPLE 8

### Synthesis of Glycerol 1-O-Tetradecylether by Acid Catalysis from Glycerol Carbonate

In a 50-mL three-necked flask, provided with a coolant and a nitrogen inlet, 8.57 g (40 mmol) commercial tetradecanol and 118 mg of Amberlyst A 35 solid acid are introduced at room temperature. The reaction medium is then brought to 140° C. and 1.18 g (10 mmol) of glycerol carbonate are added over a period of one hour. The heating at 140° C. is then extended for 1 h after the end of the addition. Then, the reaction medium is brought to room temperature. Then, 20 mL of CH₂Cl₂, as well as 5 mL d′H₂O are added. The organic phase is then decanted. The aqueous phase is extracted by 2×25 mL of CH₂Cl₂. The organic phases are combined and the CH₂Cl₂ is evaporated under reduced pressure. The crude reaction product is finally purified by flash silica column chromatography (Eluent (AcOEt/cyclohexane: 1/4 to 1/1)) to give glycerol 1-O-tetradecylether with an isolated yield of 45%.

### EXAMPLE 9

### Synthesis of Ethylene Glycol 1-O-Pentylether by Acid Catalysis from Glycerol Carbonate

In a 25-mL three-necked flask, provided with a coolant and a nitrogen inlet, 3.52 g (40 mmol) of commercial pentan-1-ol and 88 mg of Amberlyst A 35 solid acid are introduced at room temperature. The reaction medium is then brought to 140° C. and 0.88 g (10 mmol) of ethylene carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 0/1 to 1/10)) to give ethylene glycol 1-O-pentyl ether with an isolated yield of 46%.

### EXAMPLE 10

### Synthesis of Ethylene Glycol 1-O-Heptyl Ether by Acid Catalysis from Glycerol Carbonate

In a 25-mL three-necked flask, provided with a coolant and a nitrogen inlet, 4.65 g (40 mmol) of commercial heptan-1-ol and 88 mg of Amberlyst A 35 solid acid are introduced at room temperature. The reaction medium is then brought to 140° C. and 0.88 g (10 mmol) of ethylene carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 0/1 to 1/10)) to give ethylene glycol 1-O-heptyl ether with an isolated yield of 43%.

### EXAMPLE 11

### Synthesis of Ethylene Glycol 1-O-Octyl Ether by Acid Catalysis from Glycerol Carbonate

In a 25-mL three-necked flask, provided with a coolant and a nitrogen inlet, 5.21 g (40 mmol) of commercial octan-1-ol and 88 mg of Amberlyst A 35 solid acid are introduced at room temperature. The reaction medium is then brought to 140° C. and 0.88 g (10 mmol) of ethylene carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 0/1 to 1/10)) to give ethylene glycol 1-O-octyl ether with an isolated yield of 42%.

### EXAMPLE 12

### Synthesis of Ethylene Glycol 1-O-Decyl Ether by Acid Catalysis from Glycerol Carbonate

In a 25-mL three-necked flask, provided with a coolant and a nitrogen inlet, 6.33 g (40 mmol) of commercial decanol and 88 mg of Amberlyst A 35 solid acid are introduced at room temperature. The reaction medium is then brought to 140° C. and 0.88 g (10 mmol) of ethylene carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 0/1 to 1/10)) to give ethylene glycol 1-O-decyl ether with an isolated yield of 37%.

### EXAMPLE 13

### Synthesis of Ethylene Glycol 1-O-Dodecyl Ether by Acid Catalysis from Glycerol Carbonate

In a 25-mL three-necked flask, provided with a coolant and a nitrogen inlet, 7.45 g (40 mmol) of commercial dodecan-1-ol and 88 mg of Amberlyst A 35 solid acid are introduced at room temperature. The reaction medium is then brought to 140° C. and 0.88 g (10 mmol) of ethylene carbonate are added over a period of one hour. The heating at 140° C. is then extended for 1 h after the end of the addition. Then, the reaction medium is brought to room temperature. Then, 10 mL of CH₂Cl₂, as well as 2 mL of H₂O are added. The organic phase is then decanted. The aqueous phase is extracted by 2×10 mL of CH₂Cl₂. The organic phases are combined and the CH₂Cl₂ is evaporated under reduced pressure. The crude reaction product is finally purified by flash silica column chromatography (Eluent (AcOEt/cyclohexane: 0/1 to 1/10)) to give ethylene glycol 1-O-dodecyl ether with an isolated yield of 28%.

### EXAMPLE 14

### Synthesis of Propylene Glycol Pentyl Ether by Acid Catalysis from Glycerol Carbonate

| | | | | |
|---|---|---|---|---|
| Entry | Catalyst | Product | Conversion | Isolated yield |
| no. | | | (%) | (%) |

In a 50-mL three-necked flask, provided with a coolant and a nitrogen inlet, 3.52 g (40 mmol) of commercial pentan-1-ol and 118 mg of Amberlyst A 35 solid acid are introduced at room temperature. The reaction medium is then brought to 140° C. and 118 mg (10 mmol) of propylene carbonate are added over a period of one hour. The heating at 140° C. is then extended for 1 h after the end of the addition. Then, the reaction medium is brought to room temperature. Then, 20 mL of CH₂Cl₂, as well as 5 mL of H₂O are added. The organic phase is then decanted. The aqueous phase is extracted by 2×25 mL of CH₂Cl₂. The organic phases are combined and the CH₂Cl₂ is evaporated under reduced pressure. The crude reaction product is finally purified by flash silica column chromatography (Eluent (AcOEt/cyclohexane: 1/4 to 1/1)) to give propylene glycol 1-O-pentyl ether with an isolated yield of 42%.

### EXAMPLE 15

### Synthesis of Propylene Glycol Heptyl Ether by Acid Catalysis from Glycerol Carbonate

In a 50-mL three-necked flask, provided with a coolant and a nitrogen inlet, 4.65 g (40 mmol) of commercial heptan-1-ol and 118 mg of Amberlyst A 35 solid acid are introduced at room temperature. The reaction medium is then brought to 140° C. and 118 mg (10 mmol) of propylene carbonate are added over a period of one hour. The heating at 140° C. is then extended for 1 h after the end of the addition. Then, the reaction medium is brought to room temperature. Then, 20 mL of CH₂Cl₂, as well as 5 mL of H₂O are added. The organic phase is then decanted. The aqueous phase is extracted by 2×25 mL of CH₂Cl₂. The organic phases are combined and the CH₂Cl₂ is evaporated under reduced pressure. The crude reaction product is finally purified by flash silica column chromatography (Eluent (AcOEt/cyclohexane: 1/4 to 1/1)) to give propylene glycol heptyl ether with an isolated yield of 40%.

The following table presents the results of the different tests performed.

| | | | | |
|---|---|---|---|---|
| 1 | Amberlyst 35 | 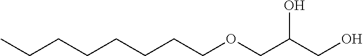 | >99 | 45 (60)* |
| 2 | Amberlyst 35 | 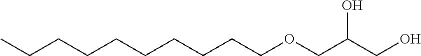 [SMILES: CCCCCCCCCCOCC(O)CO] | >99 | 46 |
| 3 | Amberlyst 35 | 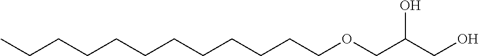 [SMILES: CCCCCCCCCCCCOCC(O)CO] | >99 | 40 |
| 4 | Amberlyst 36 | 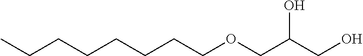 | >99 | 15* |
| 5 | Amberlyst 35 | 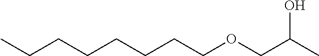 | 90 | 85* |
| 6 | Amberlyst 35 | 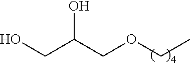 | >99 | 49 |
| 7 | Amberlyst 35 | 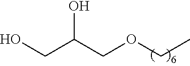 | >99 | 42 |
| 8 | Amberlyst 35 | 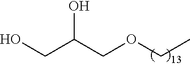 | >99 | 31 |
| 9 | Amberlyst 35 | 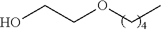 | >99 | 46 |
| 10 | Amberlyst 35 | 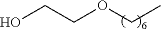 | >99 | 43 |
| 11 | Amberlyst 35 | 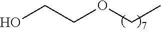 | >99 | 42 |
| 12 | Amberlyst 35 | 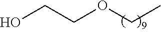 | >99 | 37 |
| 13 | Amberlyst 35 | 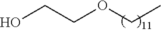 | >99 | 28 |
| 14 | Amberlyst 35 | 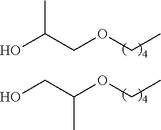 | >99 | 42 |
| 15 | Amberlyst 35 | 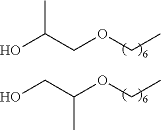 | >99 | 40 |

*GC yield

These results show that the method according to the invention enables a good conversion of glycerol carbonate and the formation of glycerol ether to be obtained.

1. Method for preparing glycerol ether or glycol ether of formula (I) and/or (I′), comprising the reaction of a compound of formula (II) with a compound of formula (III) in the presence of a heterogeneous acid catalyst 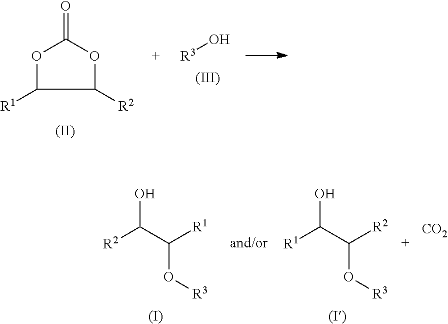
wherein
R¹ is a hydrogen atom or an alkyl radical, linear or branched, comprising 1 to 15 carbon atoms, preferably 1 to 10 carbon atoms;
R² is a hydrogen atom; an alkyl radical, linear or branched, comprising 1 to 15 carbon atoms, preferably 1 to 10 carbon atoms; or a group of formula —(CH₂)ₙOH, wherein n is an integer between 0 and 5, and n is preferably equal to 0 or 1;
R³ is an alkyl radical, linear or branched, capable of comprising one or more unsaturations, comprising 1 to 40 carbon atoms, and optionally comprising 1 or more hydroxy substituents (OH).

2. Method for preparing glycerol ether according to claim 1.

3. Method for preparing glycol ether according to claim 1.

4. Method according to claim 1, wherein the catalyst has an acid site concentration greater than or equal to 0.01 mequi/g, preferably 0.01 to 10 mequi/g, more preferably 0.01 to 6 mequi/g, and preferably 0.01 to 5 mequi/g.

5. Method according to claim 1, wherein the catalyst is a heterogeneous catalyst characterized by a Hammett constant (Ho) of −3 to −12, and preferably −5 to −12.

6. Method according to claim 1, wherein the catalyst is heterogeneous and has a specific BET surface of 5 to 500 m²/g, and preferably 10 to 100 m²/g.

7. Method according to claim 1, wherein the catalyst is chosen from the group consisting of ion exchange resins; supports impregnated with sulphuric acid, hydrochloric acid, niobic acid, hydrofluoric acid, antimony pentafluoride, heteropoly acids, triflic acid, or sulfonic acid; sulphated zirconia; zeolites, in particular zeolite Y characterized by a faujasite structure; and mixed oxides, in particular TiO₂/Al₂O₃, ReO₇/Al₂O₃, TiO₂/ZrO₂, SiO₂/Al₂O3.

8. Method according to claim 7, wherein the catalyst is chosen from the acid forms of ion exchange acid resins; supports impregnated with sulphuric acid or sulfonic acid; and sulphated zirconia.

9. Method according to claim 7, wherein the catalyst is an ion exchange acid resin bearing sulfonic groups, chosen from the resins consisting of a polystyrene skeleton bearing sulfonic groups of from the perfluorinated resins bearing sulfonic groups.

10. Method according to claim 7, wherein the ion exchange acid resin is chosen from the resins consisting of a polystyrene skeleton bearing sulfonic groups.

11. Method according to claim 1, wherein the catalyst is an ion exchange acid resin chosen from the resins consisting of a polystyrene skeleton bearing sulfonic groups and has an acid site concentration greater than or equal to 0.01 mequi/g, preferably 0.01 to 10 mequi/g, more preferably 0.01 to 6 mequi/g, and preferably 0.01 to 5 mequi/g.

12. Method according to claim 1, wherein the catalyst is an ion exchange acid resin chosen from the resins consisting of a polystyrene skeleton bearing sulfonic groups and has a Hammett constant (Ho) of −3 to −12, and preferably from −5 to −12.

13. Method according to claim 1, wherein the catalyst is an ion exchange acid resin chosen from the resins consisting of a polystyrene skeleton bearing sulfonic groups and has an acid site concentration greater than or equal to 0.01 mequi/g, preferably 0.01 to 10 mequi/g, more preferably 0.01 to 6 mequi/g, and preferably 0.01 to 5 mequi/g and has a Hammett constant (Ho) of −3 to −12, and preferably −5 to −12.

14. Method according to claim 1, wherein the reaction is implemented at a temperature of 100° C. to 200° C., and preferably 100° C. to 170° C.

15. Method for obtaining a compound of formula (I) according to claim 1, wherein R¹ is a hydrogen atom, and R² is CH₂OH.

16. Method according to claim 1, wherein the catalyst is used in proportions of 2% to 40%, preferably 5% to 20% by weight with respect to the weight of the compound of formula (II).

17. Method according to claim 1, wherein the molar ratio of formula (II) compound/formula (III) compound is 1/1 to 1/5, and preferably 1/2 to 1/4.

18. Method according to claim 1, comprising a preliminary step of preparing the compound of formula (II) by reaction between a compound of formula (IV) and carbon dioxide, in the presence of a lanthanide-based catalyst; 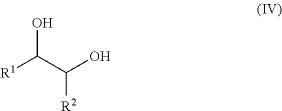
wherein R¹ and R² are as defined in claim 1.
